(19) United States
(12) Patent Application Publication
(10) Pub. No.: US 2010/0016582 A1
(43) Pub. Date: Jan. 21, 2010

(54) METHODS OF CONVERTING POLYMORPHIC FORM B OF BAZEDOXIFENE ACETATE TO POLYMORPHIC FORM A OF BAZEDOXIFENE ACETATE

(75) Inventors: Giorgio SORIATO, Verona (IT); Livius COTARCA, Cervignano del Friuli (IT); Roberto BRESCELLO, Abano Terme (IT)

Correspondence Address: WilmerHale/Wyeth LLC, 60 STATE STREET, BOSTON, MA, 02109, US

(73) Assignee: Wyeth, Collegeville, PA (US)

(21) Appl. No.: 12/369,315

(22) Filed: Feb. 11, 2009

Related U.S. Application Data
(60) Provisional application No. 61/027,607, filed on Feb. 11, 2008.

(51) Int. Cl. C07D 403/12

(52) U.S. Cl. 540/602

(57) ABSTRACT

The present invention relates to methods of preparing polymorphic Form A of bazedoxifene acetate and polymorphic Form A prepared by such methods.

### CROSS REFERENCE TO RELATED APPLICATION

This application claims the benefit of priority under 35 U.S.C. §119(e) to U.S. Patent Application Ser. No. 61/027,607 filed Feb. 11, 2008, which is hereby incorporated by reference in its entirety.

### FIELD OF THE INVENTION

The present invention is directed to methods of converting polymorphic Form B of bazedoxifene acetate to polymorphic Form A of bazedoxifene acetate as well as polymorphic Form A of bazedoxifene acetate prepared by such methods.

### BACKGROUND OF THE INVENTION

Bazedoxifene acetate has a chemical name of (1-[4-(2-azepan-1-yl-ethoxy)-benzyl]-2-(4-hydroxy-phenyl)-3-methyl-1H-indol-5-ol acetic acid) and has the chemical structure shown below:

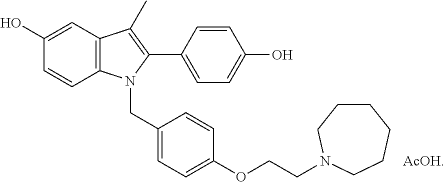

Bazedoxifene acetate belongs to the class of drugs typically referred to as selective estrogen receptor modulators (SERMs). Consistent with its classification, bazedoxifene demonstrates affinity for estrogen receptors (ER) but shows tissue selective estrogenic effects. For example, bazedoxifene acetate demonstrates little or no stimulation of uterine response in preclinical models of uterine stimulation. Conversely, bazedoxifene acetate demonstrates an estrogen agonist-like effect in preventing bone loss and reducing cholesterol in an ovariectomized rat model of osteopenia. In an MCF-7 cell line (human breast cancer cell line), bazedoxifene acetate behaves as an estrogen antagonist. These data demonstrate that bazedoxifene acetate is estrogenic on bone and cardiovascular lipid parameters and antiestrogenic on uterine and mammary tissue and thus has the potential for treating a number of different disease or disease-like states in which the estrogen receptor is involved.

U.S. Pat. Nos. 5,998,402 and 6,479,535 report the preparation of bazedoxifene acetate and characterize the salt as having a melting point of 174-178° C. The synthetic preparation of bazedoxifene acetate has also appeared in the general literature. See, for example, Miller et al., J. Med. Chem., 2001, 44, 1654-1657, which reports the salt as a crystalline solid having a melting point of 170.5-172.5° C. Further description of the drug's biological activity has also appeared in the general literature (e.g., Miller et al., Drugs of the Future, 2002, 27(2), 117-121).

It is well known that the crystalline polymorph form of a particular drug is often an important determinant of the drug's ease of preparation, stability, solubility, storage stability, ease of formulation and in vivo pharmacology. Polymorphic forms occur where the same composition of matter crystallizes in a different lattice arrangement resulting in different thermodynamic properties and stabilities specific to the particular polymorphic form. In cases where two or more polymorph substances can be produced, it is desirable to have a method to make both polymorphs in pure form. In deciding which polymorph is preferable, the numerous properties of the polymorphs must be compared and the preferred polymorph chosen based on the many physical property variables. It is entirely possible that one polymorphic form can be preferable in some circumstances where certain aspects such as ease of preparation, stability, etc. are deemed to be critical. In other situations, a different polymorph may be preferred for greater solubility and/or superior pharmacokinetics.

Polymorphic Form A of bazedoxifene acetate is disclosed in US 2005/0227965 while polymorphic Form B of bazedoxifene acetate is disclosed in US 2005/0250762. Form A has higher solubility in both aqueous and organic solvent systems than Form B. This is particularly advantageous in formulations or doses where the solubility of the particular composition is of concern. For example, higher solubility can influence bioavailability, which can affect biological absorption and distribution of the drug, as well as can facilitate formulation in liquid carriers. However, Form A is the kinetic (or meta-stable) polymorph, while Form B is the thermodynamically more stable polymorph. Form A can easily convert to Form B upon contact with a solvent or solvent mixture (e.g., ethyl acetate and ethanol), which presents a challenge to the preparation of pure Form A that is substantially free of Form B.

Because polymorphic Form A of bazedoxifene acetate provides better bioavailability in some drug formulations, there is a need for a new and practical method that can reliably produce pure polymorphic Form A of bazedoxifene acetate, including converting polymorphic Form B of bazedoxifene acetate into polymorphic Form A of bazedoxifene acetate. The methods of preparing polymorphic Form A of bazedoxifene acetate described herein help meet these and other needs. A method of preparing polymorphic Form A of bazedoxifene acetate is also disclosed in a commonly assigned and co-pending U.S. Patent Application Ser. No. 61/027,634, filed on Feb. 11, 2008.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

### SUMMARY OF THE INVENTION

In one aspect, the present invention relates to a method of preparing polymorphic Form A of bazedoxifene acetate, the method comprising:

(a) dissolving (i) polymorphic Form B of bazedoxifene acetate or (ii) a mixture of polymorphic Form A and polymorphic Form B of bazedoxifene acetate in a solvent comprising ethanol at elevated temperature to form a solution; and

(b) cool the solution to crystallize polymorphic Form A of bazedoxifene acetate.

In another aspect, the present invention relates to a method of preparing polymorphic Form A of bazedoxifene acetate, the method comprising:

(a) contacting (i) polymorphic Form B of bazedoxifene acetate or (ii) a mixture of polymorphic Form A and Form B of bazedoxifene acetate with at least one base to provide bazedoxifene free base; and

(b) treating said bazedoxifene free base with acetic acid to crystallize polymorphic Form A of bazedoxifene acetate.

In yet another aspect, the present invention relates to a method of preparing polymorphic Form A of bazedoxifene acetate, the method comprising:

(a) contacting (i) polymorphic Form B of bazedoxifene acetate or (ii) a mixture of polymorphic Form A and Form B of bazedoxifene acetate with at least one base in a solvent comprising at least one water-immiscible organic solvent and water, and in the presence of an antioxidant at elevated temperature to provide bazedoxifene free base; and

(b) treating said bazedoxifene free base with acetic acid to crystallize polymorphic Form A of bazedoxifene acetate.

In a further aspect, the present invention relates to a polymorphic Form A of bazedoxifene acetate prepared according to the methods described herein.

Other features and advantages of the invention will be apparent from the detailed description, drawings, and from the claims.

### DETAILED DESCRIPTION OF THE INVENTION

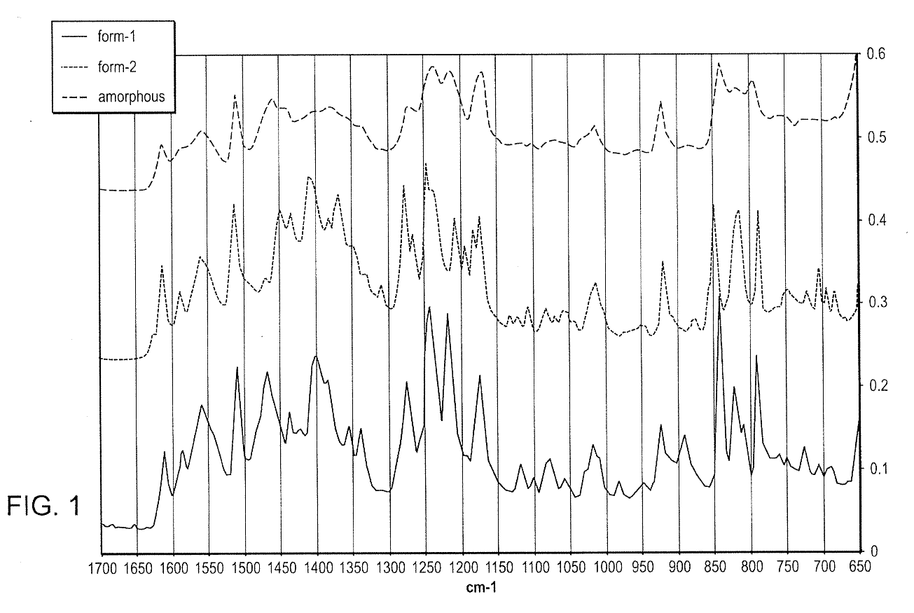

FIG. 1 shows an overlay of IR spectra of polymorphic Form A and Form B of bazedoxifene acetate (the bottom portion of FIG. 1 corresponds to Form A and the top portion corresponds to Form B).

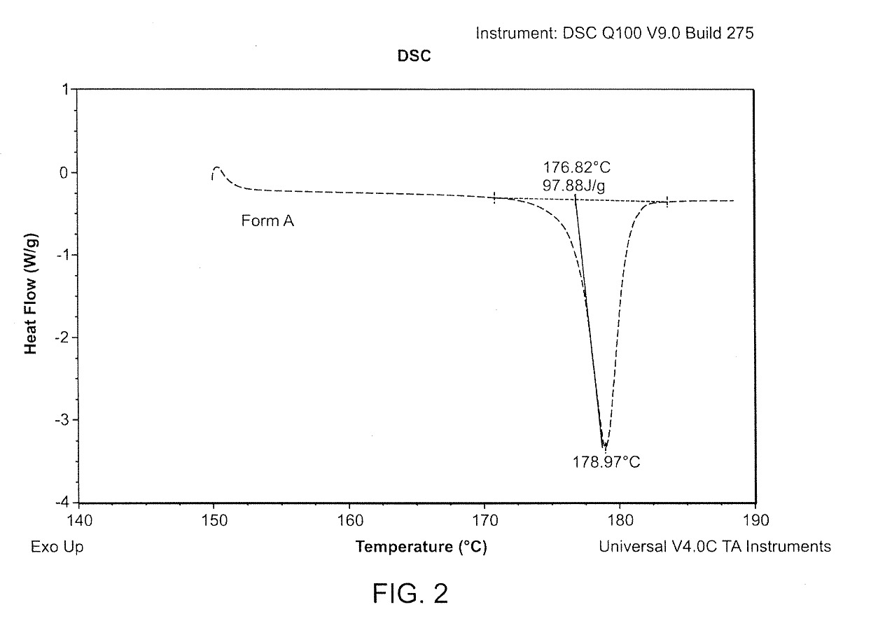

FIG. 2 shows a DSC thermogram for polymorphic Form A of bazedoxifene acetate.

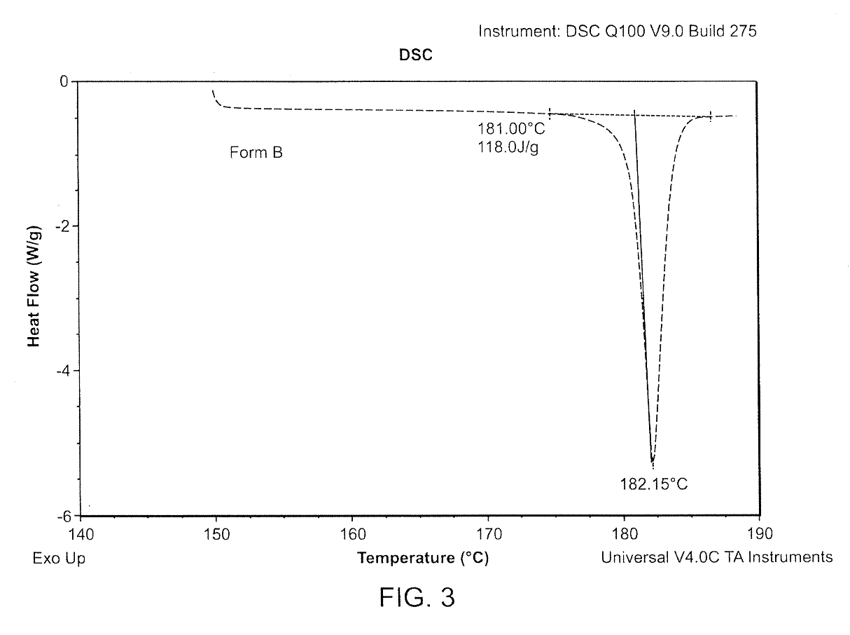

FIG. 3 shows a DSC thermogram for polymorphic Form B of bazedoxifene acetate.

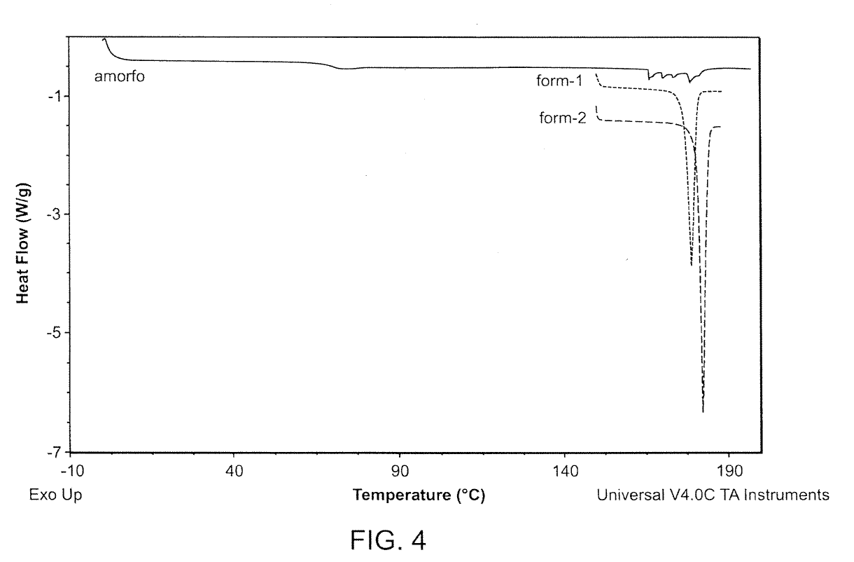

FIG. 4 shows an overlay of DSC thermograms for polymorphic Form A and Form B of bazedoxifene acetate.

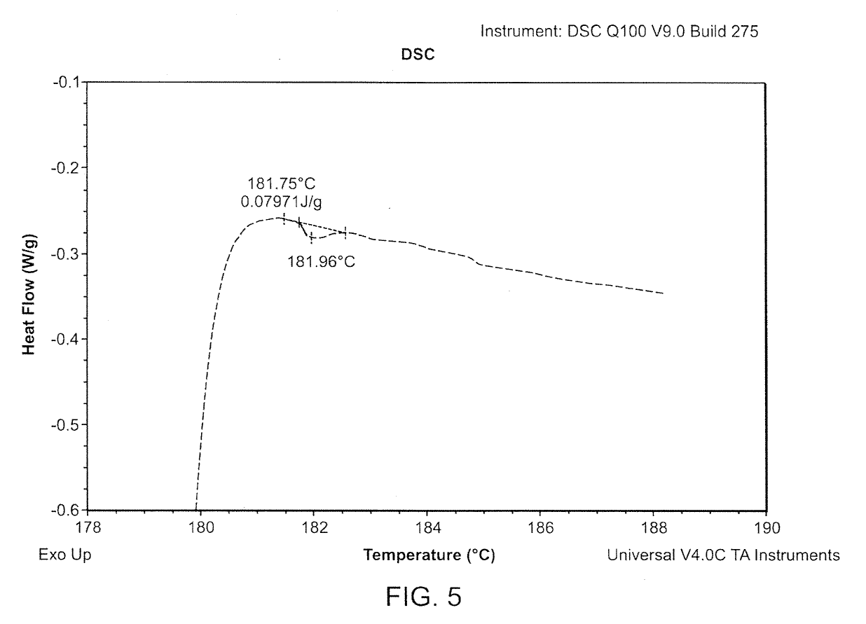

FIG. 5 is a DSC thermogram for polymorphic Form A of bazedoxifene acetate showing an example of a Form A batch with a content of Form B less than 0.1% w/w.

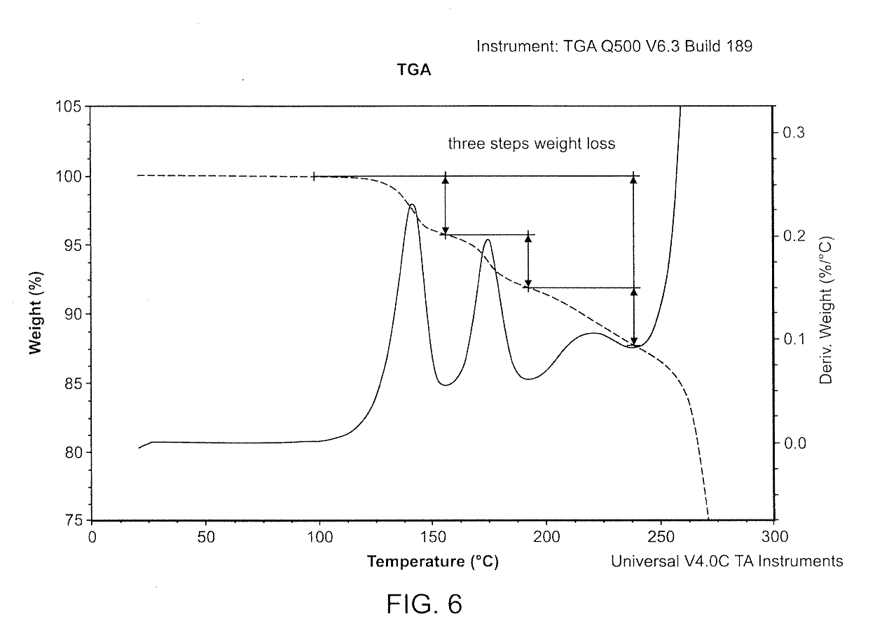

FIG. 6 shows a TGA curve for polymorphic Form A of bazedoxifene acetate.

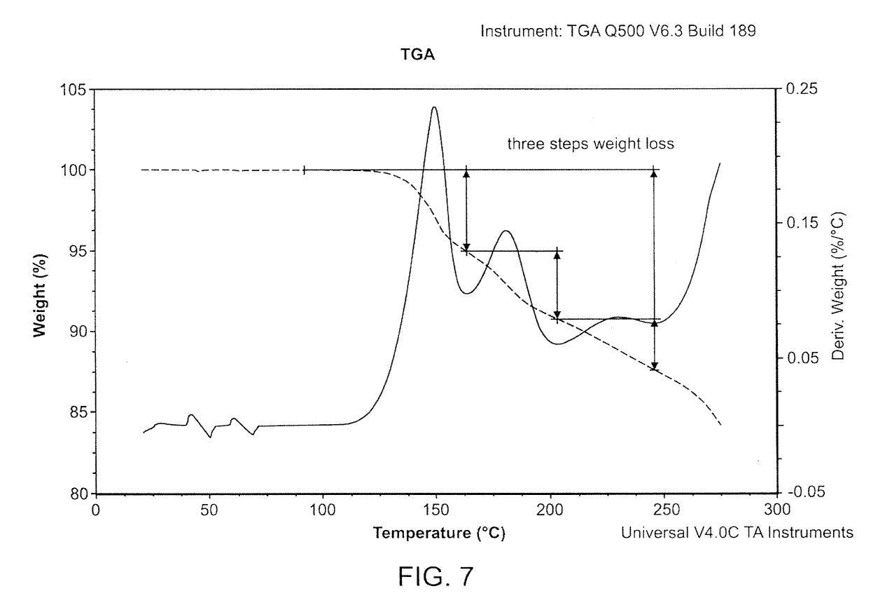

FIG. 7 shows a TGA curve for polymorphic Form B of bazedoxifene acetate.

### DETAILED DESCRIPTION OF THE INVENTION

Polymorphic Form A of bazedoxifene acetate has higher solubility in both aqueous and organic solvent systems than polymorphic Form B of bazedoxifene acetate. As a result, Form A of bazedoxifene acetate provides better bioavailability in drug formulations. However, Form A is the kinetic (or meta-stable) polymorph while Form B is the thermodynamically more stable polymorph. Form A can easily convert to Form B upon contact with a solvent or solvent mixture (e.g., ethyl acetate and ethanol), which presents a challenge to the preparation of pure Form A that is substantially free of Form B. The present invention meets this challenge by providing methods of preparing pure Form A by converting polymorphic Form B of bazedoxifene acetate or a mixture of polymorphic Form A and Form B of bazedoxifene acetate into the desired pure Form A.

The term “substantially free” as used herein refers to a compound that contains less than 10% of an undesired compound or impurity, preferably less than 5% of an undesired compound or impurity, and more preferably less than 1% of an undesired compound or impurity, e.g., less than 0.5% or less than 0.1% of an undesired compound or impurity. For example, “pure Form A that is substantially free of Form B” refers to Form A which contains less than 10% of Form B, preferably less than 5% of Form B, and more preferably less than 1% of Form B, e.g., less than 0.5% of Form B or less than 0.1% of Form B.

In one aspect, the present invention provides a method of preparing polymorphic Form A of bazedoxifene acetate, the method comprising:

(a) dissolving (i) polymorphic Form B of bazedoxifene acetate or (ii) a mixture of polymorphic Form A and polymorphic Form B of bazedoxifene acetate in a solvent comprising ethanol at elevated temperature to form a solution; and
(b) cool the solution to crystallize polymorphic Form A of bazedoxifene acetate.

In certain embodiments, in step (a) of the method described herein, said elevated temperature is at about 50° C. or higher (up to about 78° C., e.g., about 75° C., about 70° C., about 65° C., about 60° C., or about 55° C.). In certain other embodiments, said elevated temperature is at about 60° C. or higher (up to about 78° C., e.g., about 75° C., about 70° C., or about 65° C.). In yet other embodiments, said elevated temperature is at about 70° C. or higher (up to about 78° C., e.g., about 75° C.). In yet other embodiments, said elevated temperature is between about 50° to about 78° C. In yet other embodiments, said elevated temperature is between about 60° to about 78° C. In yet other embodiments, said elevated temperature is between about 70° to about 78° C. In yet another embodiment, said elevated temperature is at about 78° C.

In certain other embodiments, step (a) of the method described herein is conducted in the presence of an antioxidant. In some cases, the antioxidant is selected from ascorbic acid, sodium ascorbate, ascorbyl palmitate, citric acid, propyl gallate, alpha Tocopherol (vitamin E), vitamin E TPGS, vitamin E acetate, butylated hydroxytoluene, butylated hydroxyanisole and mixtures thereof. In another embodiment, the antioxidant is ascorbic acid.

In certain embodiments, in step (a) of the method described herein, the solvent is ethanol denatured with at least one solvent selected from the group consisting of ethyl acetate, acetone, and cyclohexane. In one embodiment, the solvent comprises ethanol denatured with acetone and cyclohexane.

In certain embodiments, in step (b) of the method described herein, the solution is cooled to about 30° C. or lower (as low as about −10° C., e.g., 25° C., 20° C., 15° C., 10° C., 5° C., 0° C., −2° C. or −5° C.). In certain other embodiments, the solution is cooled to between about −10° C. to about 30° C. In yet other embodiments, the solution is cooled to between about 0° C. to about 30° C. In yet other embodiments, the solution is cooled gradually to about −10° C. In yet other embodiments, the solution is cooled gradually to about 0° C.

In certain other embodiments, steps (a) through (b) of the method described herein are conducted under inert atmosphere. Non-limiting examples of inert atmosphere include nitrogen, argon, and so forth.

In certain embodiments, in step (b) of the method described herein, crystallization is facilitated by seeding with polymorphic Form A of bazedoxifene acetate.

In another aspect, the present invention provides a method of preparing polymorphic Form A of bazedoxifene acetate, the method comprising:

(a) dissolving (i) polymorphic Form B of bazedoxifene acetate or (ii) a mixture of polymorphic Form A and polymorphic Form B of bazedoxifene acetate in a solvent comprising ethanol at elevated temperature to form a solution; and

(b) cool the solution to crystallize polymorphic Form A of bazedoxifene acetate; and

(c) isolating polymorphic Form A of bazedoxifene acetate by filtration, washing and drying.

In certain embodiments, in step (c) of the method described herein, said drying is conducted in an agitated filter dryer. An agitated filter dryer is useful in separating solids from liquid in a single vessel. Once the dryer is charged with slurry, pressure is either applied from the top of the filter dryer using a gas, such as nitrogen, or a vacuum is pulled from beneath the filter media, thereby forcing or pulling liquid through the cloth or mesh to generate a cake. Low pressures are generally used (e.g., 1 bar) to keep the cake from becoming so compressed that the crystals fuse together. The liquid exits at the bottom of the vessel. While the crystals are collecting on the filter media, the smooth edge of the agitator acts to smooth the surface of the cake so there are no crevices. The other edge of the agitator, which can rotate in both directions, might have teeth for digging into the cake to help break it up and remove it from the filter media. The cake might be broken up and washed several times to remove all trace solvents or impurities. Heat may be applied to dryer to speed up the drying process.

In certain other embodiments, in step (c) of the method described herein, said drying is conducted in a tumble dryer. The drying can be conducted in a tumble dryer under nitrogen, and/or vacuum conditions. The tumble dryer can be purchased from a variety vendors, for example, Italvacuum CRIOX in Italy.

In another aspect, the present invention provides a method of preparing polymorphic Form A of bazedoxifene acetate, the method comprising:

(a) contacting (i) polymorphic Form B of bazedoxifene acetate or (ii) a mixture of polymorphic Form A and Form B of bazedoxifene acetate with at least one base to provide bazedoxifene free base (i.e. bazedoxifene that is free of acid, e.g., acetic acid); and
(b) treating said bazedoxifene free base with acetic acid to crystallize polymorphic Form A of bazedoxifene acetate.

In certain embodiments, step (a) of the method described herein is conducted in a solvent comprising at least one water-immiscible organic solvent and water. Non-limiting examples of the water-immiscible organic solvent include esters (R¹—C(═O)O—R², wherein R¹ and R² are each independently selected from C₁-C₄ alkyl; e.g., ethyl acetate), alkyl ethers (R¹—O—R², wherein R¹ and R² are each independently selected from C₁-C₄ alkyl; e.g., diethyl ether, methyl ethyl ether, methyl isopropyl ether, etc.), chloroform, methylene chloride and mixtures thereof. In one embodiment, the water-immiscible organic solvent is an ester. In another embodiment, the water-immiscible organic solvent is ethyl acetate.

In certain other embodiments, in step (a) of the method described herein, the at least one base is an inorganic base. In one embodiment, the inorganic base is a hydroxide of alkali metals and alkaline earth metals. In another embodiment, the inorganic base is a carbonate of alkali metals and alkaline earth metals. In yet another embodiment, the inorganic base is a bicarbonate of alkali metals and alkaline earth metals. Non-limiting examples of the inorganic base include LiOH, NaOH, KOH, Na₂CO₃, K₂CO₃, NaHCO₃, KHCO₃ and mixtures thereof.

In certain embodiments, step (a) of the method described herein is conducted at elevated temperature. In certain embodiments, the elevated temperature is at about 40° C. or higher (up to about 78° C., e.g., about 75° C., about 70° C., about 65° C., about 60° C., or about 55° C., about 50° C., or about 45° C.). In certain other embodiment, the elevated temperature is at about 50° C. or higher (up to about 78° C., e.g., about 75° C., about 70° C., about 65° C., about 60° C., or about 55° C.). In yet other embodiments, said elevated temperature is at about 60° C. or higher (up to about 78° C., e.g., about 75° C., about 70° C., or about 65° C.). In yet other embodiments, said elevated temperature is at a range from about 40° to about 78° C. In yet other embodiments, said elevated temperature is at a range from about 60° to about 78° C. In yet other embodiments, the elevated temperature is at a range from about 45° C. to about 60° C.

In certain other embodiments, step (a) of the method described herein is conducted in the presence of an antioxidant. In some cases, the antioxidant is selected from ascorbic acid, sodium ascorbate, ascorbyl palmitate, citric acid, propyl gallate, alpha Tocopherol (vitamin E), vitamin E TPGS, vitamin E acetate, butylated hydroxytoluene, butylated hydroxyanisole and mixtures thereof. In another embodiment, the antioxidant is ascorbic acid.

In certain embodiments, step (a) is conducted under inert atmosphere. Non-limiting examples include nitrogen, argon, etc.

In certain other embodiments, in step (b) of the method described herein, crystallization is facilitated by seeding with polymorphic Form A of bazedoxifene acetate.

In yet another aspect, the present invention provides a method of preparing polymorphic Form A of bazedoxifene acetate, the method comprising:

(a) contacting (i) polymorphic Form B of bazedoxifene acetate or (ii) a mixture of polymorphic Form A and Form B of bazedoxifene acetate with at least one base to provide bazedoxifene free base;

(b) treating said bazedoxifene free base with acetic acid to crystallize polymorphic Form A of bazedoxifene acetate; and

(c) isolating polymorphic Form A of bazedoxifene acetate by filtration, washing and drying.

In certain embodiments, in step (c) of the method described herein, said drying is conducted in an agitated filter dryer. In certain other embodiments, in step (c) of the method described herein, said drying is conducted in a tumble dryer. Both the agitated filter dryer and the tumble dryer are described as hereinabove.

In a further aspect, the present invention provides a method of preparing polymorphic Form A of bazedoxifene acetate, the method comprising:

(a) contacting (i) polymorphic Form B of bazedoxifene acetate or (ii) a mixture of polymorphic Form A and Form B of bazedoxifene acetate with at least one base in a solvent comprising at least one water-immiscible organic solvent and water, and in the presence of an antioxidant at elevated temperature to provide bazedoxifene free base; and

(b) treating said bazedoxifene free base with acetic acid to crystallize polymorphic Form A of bazedoxifene acetate.

In certain embodiments, in step (a) of the method described herein, the water-immiscible organic solvent is an ester such as ethyl acetate. In certain other embodiments, in step (a) of the method described herein, the at least one base is an inorganic base as described hereinabove. In yet other embodiments, the inorganic base includes LiOH, NaOH, KOH, Na₂CO₃, K₂CO₃, NaHCO₃, KHCO₃ and mixtures thereof. In certain embodiments, in step (a) of the method described herein, the antioxidant includes ascorbic acid, sodium ascorbate, ascorbyl palmitate, citric acid, propyl gallate, alpha Tocopherol (vitamin E), vitamin E TPGS, vitamin E acetate, butylated hydroxytoluene, butylated hydroxyanisole and mixtures thereof. In one embodiment, the antioxidant is ascorbic acid.

In certain embodiments, step (a) of the method described herein is conducted at elevated temperature, for example, from about 40° to about 78° C., from about 50° to about 78° C., from about 60° to about 78° C., or from about 45° C. to about 60° C. In certain other embodiments, in step (b) of the method described herein, at least one additional solvent is used to crystallize polymorphic Form A of bazedoxifene acetate. In one embodiment, said at least one additional solvent is ethanol. In another embodiment, said at least one additional solvent includes ethanol and toluene.

In certain embodiments, step (b) of the method described herein is facilitated by seeding with polymorphic Form A of bazedoxifene acetate. In certain other embodiments, step (a) of the method described herein is conducted under inert atmosphere. In certain embodiments, both step (a) and step (b) are conducted under inert atmosphere. Non-limiting examples of inert atmosphere include nitrogen, argon, etc.

In one aspect, the present invention provides a method of preparing polymorphic Form A of bazedoxifene acetate, the method comprising:

(a) contacting (i) polymorphic Form B of bazedoxifene acetate or (ii) a mixture of polymorphic Form A and Form B of bazedoxifene acetate with at least one base in a solvent comprising at least one water-immiscible organic solvent and water, and in the presence of an antioxidant at elevated temperature to provide bazedoxifene free base;
(b) treating said bazedoxifene free base with acetic acid to crystallize polymorphic Form A of bazedoxifene acetate; and
(c) isolating polymorphic Form A of bazedoxifene acetate by filtration, washing and drying.

In certain embodiments, in step (c) of the method described herein, said drying is conducted in an agitated filter dryer. In certain other embodiments, in step (c) of the method described herein, said drying is conducted in a tumble dryer. Both the agitated filter dryer and the tumble dryer are described as herein.

In another aspect, the present invention is directed to polymorphic Form A of bazedoxifene acetate prepared according to the methods described herein. In certain embodiments, the polymorphic Form A of bazedoxifene acetate prepared according to such methods is more than 99% w/w pure, for example, more than 99.5% w/w or more than 99.9% w/w pure. In certain other embodiments, the polymorphic Form A of bazedoxifene acetate contains less than 1% w/w of Form B, for example, less than 0.5% w/w or 0.1% w/w Form B.

### Methods of Preparation

According to Scheme 1, polymorphic Form B of bazedoxifene acetate or a mixture of polymorphic Form A and polymorphic Form B of bazedoxifene acetate is dissolved in a solvent, such as ethanol at elevated temperature, to form a solution. The solution is then cooled slowly to crystallize Form A of bazedoxifene acetate. Optionally, before the crystallization step, the solution can be passed through a filtration device such as Tonsil Optimum L80FF bed suitable for the adsorption of impurities.

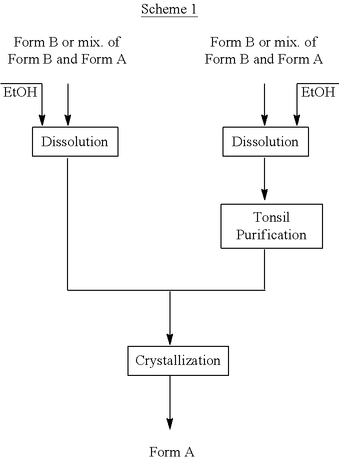

Alternatively, bazedoxifene acetate (Form B or a mixture of Form B and Form A) can be treated with at least one base (e.g., LiOH, NaOH, KOH, Na₂CO₃, K₂CO₃, NaHCO₃, and KHCO₃) to produce bazedoxifene acetate free base, followed by treatment with acetic acid and crystallization.

The invention is further illustrated by the following examples. The examples are provided for illustrative purposes only. They are not to be construed as limiting the scope or content of the invention in any way.

### EXAMPLES

### Example 1

### Infrared (IR) Analysis of Polymorphic Form A and Form B of Bazedoxifene Acetate

The solid state of both polymorphic Form A and Form B of Bazedoxifene Acetate has a characteristic Infrared (IR) spectrum. The transformation from Form A to Form B can be followed “on-line” by FT-IR/ATR and FBRM technology (Lasentec probe). The “IR on-line” experiments can be carried out using the system React-IR 4000 in the configuration that uses the probe with diamond sensor ATR (Dicomp). The “size-distribution on line” experiments are executed by using the system Lasentec FBRM equipped with D600R probe.

IR measures are carried out opportunely placing a few mg of the powder of the sample on the diamond sensor ATR of the probe. The inquired spectral interval is 4000-650 cm-1 with 4 cm-1 resolution (128 scans for every collected spectrum). Some experiments are executed creating an ethanol vapor atmosphere around the ATR sensor avoiding the direct contact between the liquid solvent and the powder of bazedoxifene acetate.

Some characteristic absorption peaks of the polymorphic Form A and Form B of bazedoxifene acetate are shown in Table 1. FIG. 1 shows an overlay of IR spectra of polymorphic Form A and Form B of bazedoxifene acetate. The bottom portion of FIG. 1 is the IR spectrum for Form A while the top portion of FIG. 1 is the IR spectrum for Form B.

TABLE 1

| | IR Wavelength number (cm⁻¹) | | | |
|---|---|---|---|---|
| Form A | 841 | 1242 | 1356 | 1466 |
| Form B | 849 | 1207 | 1368 | 1449 |

### Example 2

### Differential Scanning Calorimetry (DSC) Analysis of Polymorphic Form A and Form B of Bazedoxifene Acetate

Differential scanning calorimetry (DSC) is conducted with aluminum hermetic pans, generally in the temperature interval of 150-190° C. at 5° C./min. Form A and Form B can be distinguished by their respective melting point. The DSC results are summarized in Table 2. FIG. 2 is a DSC thermogram for polymorphic Form A of bazedoxifene acetate. FIG. 3 is a DSC thermogram for polymorphic Form B of bazedoxifene acetate. FIG. 4 shows an overlay of DSC thermograms for polymorphic Form A and Form B of bazedoxifene acetate.

TABLE 2

| Solid state | Onset (° C.)* | Peak (° C.)* | Energy (J/g)* |
|---|---|---|---|
| Form A | 176.0-177.0 | 178.0-179.0 | Endo 95-100 |
| Form B | 180.5-181.5 | 182.0-182.8 | Endo 115-120 |

*Medium values from repeated analysis on samples considered pure polymorph and on HPLC specification.

The DSC technique can be used to quantitatively determine the presence of polymorphic Form B of bazedoxifene acetate in batches produced as polymorphic Form A of bazedoxifene acetate. FIG. 5 shows an example of a Form A batch with a content of Form B less than 0.1% w/w.

### Example 3

### Thermogravimetric Analysis (TGA) of Polymorphic Form A and Form B of Bazedoxifene Acetate

Thermogravimetric Analysis (TGA) is conducted with a platinum pan in the temperature range of 25°-280° C. at 4° C./min. in modality “High-Res TGA”. During the heating (in an open system), polymorphic Form A and Form B of bazedoxifene acetate release acetic acid above 100° C., and goes to completion generally between 235° and 245° C. The amount of the weight loss is between 11 and 12% and correspond to acetic acid of neutralization (theoretical 10.8%). The TGA profile of the acetic acid loss of Form A and Form B is subdivided into three fractions. The quantity of each fraction is characteristic of every single batch. FIG. 6 shows a TGA curve for polymorphic Form A of bazedoxifene acetate. FIG. 7 shows a TGA curve for polymorphic Form B of bazedoxifene acetate.

### Example 4

### Preparation of Bazedoxifene Acetate Form A from Form B

A reactor vessel kept under nitrogen is charged with ethanol denatured with cyclohexane-acetone (423.8 Kg, 536.4 L), ascorbic acid (0.26 Kg), polymorphic Form B of bazedoxifene acetate (26.4 Kg) and acetic acid (0.75 Kg, 0.71 L). The reaction mixture is degassed and then heated at reflux temperature until complete dissolution of the reaction mixture. Then the solution is cooled to about 40°-45° C. Active charcoal Anticromos standard E (2.0 Kg) is charged and after about 60 minutes of stirring at about 45° C., the charcoal-treated slurry is filtered on a plate filter. The filtration equipment is gently heated before and during the operation by rinsing with about 40.0 Kg (50.6 L) of denatured ethanol preheated at about 60° C. (before) and with a jacket water flow at about 60° C. The plate filter is prepared before use by passing slurry of 0.4 Kg of Celite and 50.0 Kg (63.3 L) of denatured ethanol. The filter jacket water flow is maintained at about 40° C. After the filtration is complete, the solid mass is then washed with 31.6 Kg (40.0 L) of pre-heated denatured ethanol. The washing filtrate maintained at about 40° C. is transferred through the plate filter previously used. The filtrate is then cooled at about 30° C. in about 30 minutes and seeded with 0.1 Kg of polymorphic Form A of bazedoxifene acetate. The temperature of the reaction mixture is gradually decreased to about −10° C. and the crystalline product is centrifuged, followed by washing and drying to afford about 18-19 Kg of polymorphic Form A of bazedoxifene acetate, with purity at no less than 99%.

### Example 5

### Preparation of Bazedoxifene Acetate Form A from a Mixture of Form B and Form A

In a reactor flushed with nitrogen, 1500 g of a mixture of bazedoxifene acetate Form A and Form B (Form A contaminated with Form B), 14.2 g of ascorbic acid, 10937 g of ethyl acetate, 251.6 g of sodium bicarbonate and 3500 g of demineralized water are charged. The mixture is heated to about 60° C. in about 1 hour under nitrogen. After about 1 hour of maintaining at that temperature, the reaction mixture is cooled to about 40°-45° C. and left at that temperature for about 30 minutes The aqueous phase (ca. 3400 g) is separated. The organic phase is treated under stirring with 1000 g of demineralized water. After about 30 min, the aqueous phase (ca. 990 g) is separated and then 14.2 g of ascorbic acid and 43 g of celite are added. The solvent is distilled off at about 45°-50° C. under reduced pressure until the internal reaction volume reaches to about 4550-6000 mL. The temperature is then lowered to about 30° C. and 6537 g of ethanol and 520 g of toluene are charged. The reaction mixture is heated under nitrogen at about 48°-50° C. and stirring for about 30 minutes and then filtered. Then 57.1 g of acetic acid and 2.8 g of bazedoxifene acetate Form A are added to start crystallization. A second portion of 197.1 g of acetic acid is added and after cooling to about −2°-0° C. and maintaining at that temperature for about 2 hours, the slurry is filtered and washed with 1037 g of ethanol (pre-cooled at about −2°-0° C.). Ca. 1480 g of wet bazedoxifene acetate Form A is obtained. After drying at about 45-50° C. under reduced pressure, ca. 1339 g of pure bazedoxifene acetate Form A are obtained (Yield 90.5%).

Table 3 below shows the analytical results of three batches (Experiment Nos. 5.1, 5.2 and 5.3) that describe the purity of bazedoxifene acetate Form A following the general procedure of Example 5.

TABLE 3

| Experiment No. | 5.1 | 5.2 | 5.3 |
|---|---|---|---|
| Form B Content (%) of | 11% | 5.2% | 58% |
| the Starting Material | | | |
| Form B Content (%) of | 0% | 0% | 0% |
| the Final Product | | | |

We claim:

1. A method of preparing polymorphic Form A of bazedoxifene acetate, the method comprising:
(a) dissolving (i) polymorphic Form B of bazedoxifene acetate or (ii) a mixture of polymorphic Form A and polymorphic Form B of bazedoxifene acetate in a solvent comprising ethanol at elevated temperature to form a solution; and
(b) cool the solution to crystallize polymorphic Form A of bazedoxifene acetate.

2. The method of claim 1, wherein in step (a), said elevated temperature is at about 60° C. or higher.

3. The method of claim 1, wherein step (a) is conducted in the presence of an antioxidant.

4. The method of claim 3, wherein the antioxidant is selected from ascorbic acid, sodium ascorbate, ascorbyl palmitate, citric acid, propyl gallate, alpha Tocopherol (vitamin E), vitamin E TPGS, vitamin E acetate, butylated hydroxytoluene, butylated hydroxyanisole and mixtures thereof.

5. The method of claim 3, wherein the antioxidant is ascorbic acid.

6. The method of claim 1, wherein in step (b), the solution is cooled to about 30° C. or lower.

7. The method of claim 1, wherein in step (a), the solvent is ethanol denatured with at least one solvent selected from the group consisting of ethyl acetate, acetone, and cyclohexane.

8. The method of claim 1, further comprising isolating polymorphic Form A of bazedoxifene acetate by filtration, washing and drying.

9. A method of preparing polymorphic Form A of bazedoxifene acetate, the method comprising:
(a) contacting (i) polymorphic Form B of bazedoxifene acetate or (ii) a mixture of polymorphic Form A and Form B of bazedoxifene acetate with at least one base to provide bazedoxifene free base; and
(b) treating said bazedoxifene free base with acetic acid to crystallize polymorphic Form A of bazedoxifene acetate.

10. The method of claim 9, wherein step (a) is conducted in a solvent comprising at least one water-immiscible organic solvent and water.

11. The method of claim 10, wherein the water-immiscible organic solvent is ethyl acetate.

12. The method of claim 9, wherein in step (a), the at least one base is an inorganic base.

13. The method of claim 12, wherein the inorganic base is a hydroxide of alkali metals and alkaline earth metals, a carbonate of alkali metals and alkaline earth metals, or bicarbonate of alkali metals and alkaline earth metals.

14. The method of claim 12, wherein the inorganic base is selected from the group consisting of LiOH, NaOH, KOH, Na₂CO₃, K₂CO₃, NaHCO₃, and KHCO₃.

15. The method of claim 9, wherein step (a) is conducted at elevated temperature.

16. The method of claim 15, wherein said elevated temperature is at about 40° C. or higher.

17. The method of claim 9, wherein step (a) is conducted in the presence of an antioxidant.

18. The method of claim 17, wherein the antioxidant is selected from ascorbic acid, sodium ascorbate, ascorbyl palmitate, citric acid, propyl gallate, alpha Tocopherol (vitamin E), vitamin E TPGS, vitamin E acetate, butylated hydroxytoluene, butylated hydroxyanisole and mixtures thereof.

19. The method of claim 17, wherein the antioxidant is ascorbic acid.

20. The method of claim 9, wherein in step (b), at least one additional solvent is used to crystallize polymorphic Form A of bazedoxifene acetate.

21. The method of claim 20, wherein said at least one additional solvent is ethanol and/or toluene.

22. The method of claim 9, further comprising isolating polymorphic Form A of bazedoxifene acetate by filtration, washing and drying.

23. A method of preparing polymorphic Form A of bazedoxifene acetate, the method comprising:
(a) contacting (i) polymorphic Form B of bazedoxifene acetate or (ii) a mixture of polymorphic Form A and Form B of bazedoxifene acetate with at least one base in a solvent comprising at least one water-immiscible organic solvent and water, and in the presence of an antioxidant at elevated temperature to provide bazedoxifene free base; and
(b) treating said bazedoxifene free base with acetic acid to crystallize polymorphic Form A of bazedoxifene acetate.

24. The method of claim 23, wherein the water-immiscible organic solvent is ethyl acetate.

25. The method of claim 23, wherein the at least one base is an inorganic base selected from the group consisting of LiOH, NaOH, KOH, Na₂CO₃, K₂CO₃, NaHCO₃, and KHCO₃.

26. The method of claim 23, wherein the antioxidant is ascorbic acid.

27. The method of claim 23, wherein step (a) is conducted at elevated temperature from about 45° C. to about 60° C.

28. The method of claim 23, wherein in step (b), at least one additional solvent is used to crystallize polymorphic Form A of bazedoxifene acetate.

29. The method of claim 28, wherein said at least one additional solvent is ethanol and/or toluene.

30. The method of claim 23, further comprising isolating polymorphic Form A of bazedoxifene acetate by filtration, washing and drying.
